(19) United States
(12) Patent Application Publication
(10) Pub. No.: US 2022/0157457 A1
(43) Pub. Date: May 19, 2022

(54) AN INTEGRATED HEALTH AND SECURITY MANAGEMENT SYSTEM

(71) Applicant: C. Rafin & Co Pty Ltd, Wollongong, New South Wales (AU)

(72) Inventors: Van Tam Phan, Wollongong, New South Wales (AU); Victor Belina Stawski, Cringila, New South Wales (AU); Matthew Macbeth, Golden Beach, Queensland (AU); Alejandro Duque Millan, Lindisfarne, Tasmania (AU)

(21) Appl. No.: 17/593,203

(22) PCT Filed: Mar. 13, 2020
(86) PCT No.: PCT/AU2020/050243

§ 371 Date: Sep. 10, 2021

(30) Foreign Application Priority Data
Mar. 13, 2019 (AU) 2019900838
Mar. 13, 2019 (AU) 2019900839

(51) Int. Cl. G16H 40/67; H04W 12/64; G08B 21/04; G08B 25/01; H04W 4/90; H04W 4/029

(52) U.S. Cl. CPC G16H 40/67; H04W 12/64; H04W 4/029; G08B 25/016; H04W 4/90; G08B 21/0453

(57) ABSTRACT

Disclosed is a mobile client care device, comprising a housing mounting circuitry including a processor arranged to execute computer processes, an alarm function process arranged to implement an alarm indication, the communications arrangement comprising a local communications process arranged to locate any available devices having a compatible local communications process, and communicate the alarm to the available device, whereby the available device may communicate with a remote location.

### TECHNICAL FIELD

The present invention relates to a system and method for facilitating patient care and security, and particularly, but not exclusively, to a system and method for facilitating patient care and security remotely.

The present invention also relates to systems, devices, and methods for client care applications, and particularly, but not exclusively, to communications arrangements of systems and devices for client care applications.

### BACKGROUND ART

Health management has traditionally been carried out in institutions such as hospitals. There is an increasing trend today, however, particularly in developed countries where there is an aging demographic, to managing people in their own homes. Home care may be facilitated by organisations that provide care in the community. This may be done by the provision of medical professionals, such as community nurses, who visit patients at home.

It is also known to use technology to facilitate personal security in the home, particularly for aged, infirm and disabled persons. For example, “personal alarm” devices are available to enable a person to alert a remote operator if they are in an alarm situation. For example, they may have fallen down and been hurt, and they may alert the remote operator by actuating their alarm device (which may be a wearable device).

Such Personal Emergency Response Systems (PERS) are implemented using hardware specifically designed for the PERS function, usually separate from any other security systems or any other systems in the home/residence. There are in the order of half a million PERS's currently in Australia, each having purpose-designed hardware and software.

The Applicant has developed a comprehensive in-home health and security system, described in their earlier International patent application PCT/AU2013/000626. The contents of that application are incorporated herein by reference.

While there are some systems available for managing health and security within the home, there are no comprehensive systems available for monitoring patients outside the home.

There are available simple applications that enable the implementation of a PERS on a mobile phone, for example, but those are generally limited to manually activatable alarm applications.

It is to be understood that, if any prior art is referred to herein, such reference does not constitute an admission that the prior art forms a part of the common general knowledge in the art, in Australia or any other country.

### SUMMARY

The present invention provides a mobile application and system that may be used in and out of the home, and that implements a care function. The care function may include personal security and also health parameter monitoring.

The mobile application may also enable communication between patients and carers, such as health professionals, over the system.

In a first aspect, there is provided a system for health and security management, including:
a mobile client application comprising executable codes downloadable to a client's device, the client application comprising a messaging module adapted to transmit messages to one or more recipient devices associated with approved users who are associated with the client, and to receive messages transmitted from the one or more recipient devices;
a data transmission and receiving module adapted to receive a data from one or more peripheral devices in communication with the client device or one or more instruments embedded within the client device, and transmit the data to a backend system;
the backend system being adapted to support a portal accessible by one or more approved users to review the data.

The messaging module may broadcast the messages to the one or more recipient devices.

The messaging module may use the Session Initiation Protocol to transmit the messages. The backend system may be configured to constant monitor the transmissions.

In an embodiment, the recipient devices are devices on which there are installed other instances of the client application, or devices which host an access interface to the backend system. For example, the recipients may be doctors or nurses who provide care to the client, who have access to recipient devices such as smart phones, tablets, or other computing devices.

The one or more recipients include a computer on which is supported an active login session on the portal supported by the backend system.

The application can include a widget which is adapted to be responsive to a voice command, to launch the mobile application.

The backend system can comprise a processor which includes or is operatively connected to an artificial intelligence module which is adapted to infer occurrences of emergencies or potential adverse events based on the data transmitted to the backend system.

The data can be assessed by the artificial intelligence module before being stored in data storage.

The mobile application can include executable code which causes a processor of the client device or of the backend system to allocate a memory location to store one or more rules, wherein the messaging module of the mobile device or a communication module of the backend system is adapted to transmit an alert message to at least one of the one or more recipients, when it receives a measurement or detection data which breaches at least one of the one or more rules.

The rules can define the ranges of expected data from the peripheral devices or instruments embedded within the client device, or the frequency or timing of certain events which the peripheral device(s) are expected to detect. For example, the peripheral device or embedded instrument may be a blood pressure sensor, a heart rate monitor, a blood glucose monitor, a movement sensor, etc.

The executable code can cause the processor to allocate a memory location to store one or both of the following: a registration information for the peripheral devices or embedded instruments; a contact list.

In an embodiment where the client device has geo-location capability, the executable code can cause the processor to allocate a memory location to store a geofence setting information including a centre and a radius defining a geographical location.

The application can comprise a plurality of executable program modules to provide a plurality of user interfaces, including one or more of the following: an emergency interface which is automatically activatable, activatable by the client, or activatable by a command via the backend system, provided by at least one of the approved users;
a geofence settings interface, which enables a definition of a geographical activity area associated with the client;
a measurement or detection devices interface adapted to allow user control of the one or more peripheral devices or embedded instruments;
a user registration interface which enables user information to be entered or updated from the client device;
an information or settings interface for displaying device and/or user setting information, and for updating device information and/or user setting information; a messaging and contact interface for creating, sending, and receiving text-based messages to one or more recipient devices, or for initiating or accepting an audio-video communication session.

The mobile application can be adapted to receive a control command from the backend system, initiated by one of the one or more approved users, for remote control of at least one of the one or more peripheral devices or embedded instruments.

The peripheral devices or embedded instruments can include one or more of the following: a geo location system, one or more vital signs sensors; one or more activity monitors to sense movements of the client; one or more motion detectors, appliances, or security apparatuses provided in a residence of the client.

The mobile application as mentioned above in the first aspect provides a comprehensive system which may be associated with a mobile device (e.g. a smartphone, smart watch, tablet, etc.). It interacts with peripheral devices and/or embedded instruments within the mobile device. The comprehensive system can be used within or outside the client's home. In embodiments where the peripheral devices include home appliances or security apparatuses equipped with connectivity, the mobile application is part of a comprehensive system which allows remote operation, or automation, of appliances or security apparatuses in the client's place of residence.

In another aspect, there is provided a health and security management system comprising a client device having installed thereon a client application as mentioned above in relation to the first aspect.

The system can comprise a plurality of the client device, in communication with a backend system.

The backend system can include at least one database which are accessible and updatable by one or more of the approved users via their respective recipient device or via a web-based portal supported by the backend system.

The at least one database can include user registration, measurement and detection data database, rules definition, past events or messages.

In another aspect, there is provided an apparatus for providing health and security management, comprising a computing device, memory, and operating system supporting computer processes, and a computer process processing a client application in accordance with the first aspect mentioned above. The device may be a mobile device.

In another aspect, there is provided a health and security management system. The system comprises a backend server having stored thereon registration details of two or more users, at least one user being a client whose health and security is being managed. The backend server is adapted to communicate over a long range communication network with two or more apparatuses in accordance with the first aspect mentioned above, at least one apparatus being a client apparatus, the other apparatuses being apparatuses of users associated with the client. The apparatuses each has a processor. At least one non-transitory computer readable medium storing machine-readable instructions for execution by the processor to: monitor, over the long range network, communication from the backend server which is to be transmitted to the two or more apparatuses; obtain data from one or more sensors, detectors, or monitors, and transmits the data over the long range communication network to the backend serer; initiate a user interface process when an alert condition is met, the alert condition being met if the obtained data is determined to breach one or more conditions or to satisfy one or more detection rules. The backend system is adapted to monitor direct communication between said apparatuses; and receive said data from the client apparatus.

The determination of whether the alert condition is met can be made by a processor of the backend server.

The processor of the client apparatus can be configured to determine if the obtained data satisfy an emergency condition, determination that the emergency condition is satisfied, communicate with at least one other apparatus using the direct communication process module.

The client apparatus can be configured to transmit an alert to the other apparatuses using the direct communication process module.

The user interface process can comprise a process to request an input from the client.

In accordance with another aspect, the present invention provides a mobile client care device, comprising a housing mounting circuitry including a processor arranged to execute computer processes, an alarm function process arranged to implement an alarm indication, a communications arrangement, comprising a local communications process arranged to locate a compatible device having a compatible local communications process, and communicate the alarm to the compatible device, whereby the compatible device may communicate with a remote location.

In an embodiment, the mobile client device may be wearable by a client (e.g. an elderly or disabled person) and the client may take it out of their home. In embodiments, the device may be in the form of a pendant, or a wrist mounted device, or any other form. In an embodiment, the local communications process is arranged to locate and contact devices that have a corresponding local communications process and also have a remote communications process. The mobile client device may therefore be able to effectively utilise the remote communications process of the available device to communicate an alarm to a remote location.

This has the advantage, in embodiments, that the mobile client care device can incorporate a battery which can last for a very long time, as the local communications process does not utilise as much power as a remote communications technology (such as mobile phone technology). This enables the client care device to be reliable and available to function, without it having to be recharged on a regular basis. In some embodiments, the battery may last a number of years.

In an embodiment, the local communications arrangement may comprise circuitry and processes implementing Bluetooth™ 4 or 5 (short range and long range). In an embodiment, the device incorporate a plurality of different local communications process (e.g. BT4, Bt5—short and long range). This advantageously provides redundancy.

In an embodiment, where the device has a plurality of local communications protocols available, it may try each in turn until it finds and connects with an available device having a compatible communications protocol.

In an embodiment, the device comprises a display, which enables messages to be conveyed to the client, so that the client may receive feedback about the alarm or other functions of the device.

In accordance with a second aspect, the present invention provides a system for client health care and security, the system comprising a plurality of devices, each of the plurality of devices comprising a communications arrangement, comprising a local communications process arranged to locate available devices having compatible local communications process, for communication, and at least one of the devices having a remote communications process for communicating remotely.

In embodiments, the devices may include devices such as PERS alarms, smoke alarms, motion sensors, home automation devices, and other devices.

In an embodiment, local communications arrangement is arranged to implement Bluetooth™ 5, both long range and short range. In embodiments, communications arrangement is also arranged to implement BT4, as well as BT5.

In embodiments, the system of plurality of devices may form an “eco-system” of devices for facilitating client health care and security. In an embodiment, communications process comprises a local transmitter/receiver communications protocol which all devices in the eco-system operate on.

### BRIEF DESCRIPTION OF THE DRAWINGS

Embodiments will now be described by way of example only, with reference to the accompanying drawings in which

FIG. 1 is a schematic diagram depicting a health and security management system in accordance with one embodiment of the invention;

FIG. 2 is an example of a menu interface of a client application of an embodiment of the invention;

FIG. 3 is an example of a contact list interface of the client application;

FIG. 4 is an example of a devices interface of the client application;

FIG. 5 is an example of a device information interface of the client application;

FIG. 6 is an example of a client information interface of the client application;

FIG. 7-1 is an example of a starting display of a geofence interface of the client application;

FIG. 7-2 is an example of an intermediate display of the geofence interface of the client application;

FIG. 7-3 is an example of the geofence interface of the client application, where two geofenced areas have been set using the interface;

FIG. 8 is an example of an initial display of the emergency interface of the client application;

FIG. 9 is an example of a subsequent display of the emergency interface of the client application;

FIG. 10 is an example of an intermediate stage of the emergency interface, asking the client to confirm that an alarm is to be sent;

FIG. 11 is an example of an initiation or launch display of the client application, where a float button and a destination area are presented for triggering an alarm;

FIG. 12 is a flow chart depicting a process which may occur when a problem event is detected; and

FIG. 13 is an example of a message display interface of the client application;

FIG. 14 is a schematic diagram of a system and devices in accordance with embodiments of the invention;

FIG. 15 is a schematic diagram of a device in accordance with an embodiment of the invention, and

FIG. 16 is a flow diagram illustrating one connection and communication process of the embodiment of FIG. 15.

### DETAILED DESCRIPTION

In the following detailed description, reference is made to accompanying drawings which form a part of the detailed description. The illustrative embodiments described in the detailed description, depicted in the drawings and defined in the claims, are not intended to be limiting. Other embodiments may be utilised and other changes may be made without departing from the spirit or scope of the subject matter presented. It will be readily understood that the aspects of the present disclosure, as generally described herein and illustrated in the drawings can be arranged, substituted, combined, separated and designed in a wide variety of different configurations, all of which are contemplated in this disclosure.

The technology has applications particularly in the areas of care, e.g. for the elderly or people living with disability, home monitoring, and smart home management. It is an advantage that much of the functionality can also be implemented outside the home. By way of example, the technology will be discussed in the following paragraphs in the context of home care and monitoring, and care and monitoring outside the home. However, the skilled person will appreciate that the technology can have other applications, which are not limited to the provision of home care or monitoring.

### Health and Security Management System

### System Structure

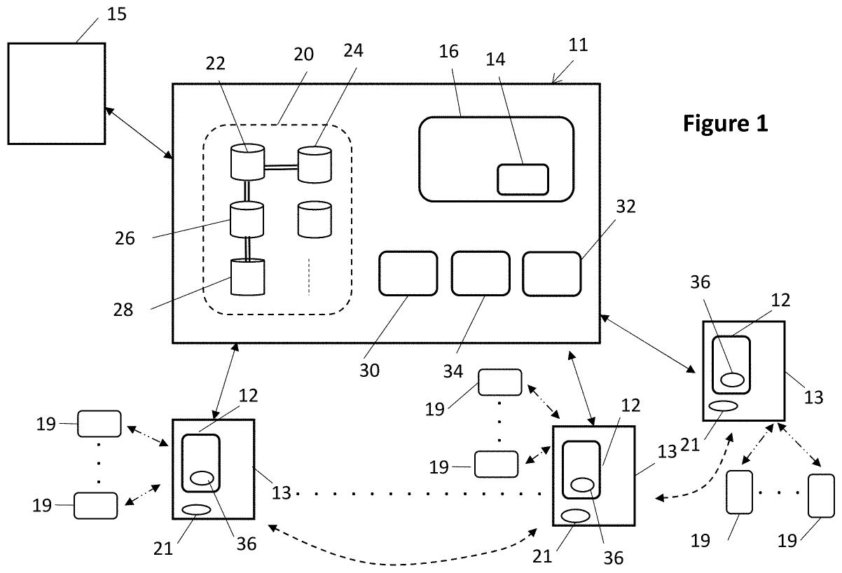

FIG. 1 depicts an example implementation of a health and security management system 10. The system 10 includes a backend system 11 which comprise a backend server. The backend system 11, as will be explained, supports one or more instances of client applications 12. Each instance of a client application is provided as a downloadable program to be installed on a client device 13. The client device is shared or personal computer, a mobile device such as a smart watch or a smart phone, or a built for purpose device. The backend system 11 also supports a web-based portal 14, which is accessible by approved users via the internet from their stationary or mobile computers 15. The users will need to verify their credentials (e.g. by providing a log-in identifier and a password) to access the portal 14.

The client application 12 is downloadable to a client device 13. It contains computer readable instructions executable by a processor of the client device, to implement one or more of the embodiments provided herein. The instructions may be implemented as program modules, i.e. functions, objects, program interfaces, memory structures, etc.

The technology disclosed herein involves bi-directional communication between various users of the system in real time, as well management and control of various devices linked to the client device, and/or installed in the client's environment. These devices are instruments 21 embedded in the client device, or they are devices 19 which are peripheral to the client device.

The management system is intended to provide an integrated healthcare, security, and/or home automation service for a client. This integrated service is enabled by the bi-directional communication between the client and other users, in conjunction with the various features of the management system. The other users are connected to the service via web portals or their own “client devices” on which the client application is installed. The web portals presented may differ depending on the user. For instance, an approved user who is a family member.

The management system is adapted to allow bidirectional communication audio and/or video, preferably both audio and video, between the backend system 11 and the client devices 13 or a computer 15 on which an approved user is accessing the portal 14. The management system further allows the broadcast of text, or preferably audio-video, messages directly between the client devices 13. This communication is preferably encrypted, e.g. by the client applications, for security and privacy purposes.

Preferably, the client application 12 is adapted to constantly communicate with the backend system 11 in two directions.

This enables the backend system 11 to monitor an array of system data, status indicators, or potential faults. Examples include but are not limited to: connection status with the client device, battery status of the client device or peripheral devices, device fault signals, software versions or update status. In an embodiment, where the backend system 11 detects a potential fault (e.g. ceases to have satisfactory connection quality with a client device, or detects a low battery status in the client device), it will issue a message to the client device of the client and/or one or more approved user.

Similarly, the client device 13 is adapted to constantly, or substantially constantly, “listen to” the backend system 11, for messages which can be transmitted (e.g. broadcast) to at least one client device. The messages may include alert messages (e.g. activity monitoring alerts), or notification messages such as reminders for appointments, informational messages, software updates, or other transmission of event messages (e.g. a change in a concierge service provided by the integrated service manager).

The communication data is encoded to be transmitted, e.g. broadcast, using a direct messaging system within the client application, using a communication protocol, such as Session Initiation Protocol (SIP). The communication between client devices (devices on which the client applications are installed) is not required to be supported by the backend server. Although there is direct communication between client applications, the backend system can also optionally control the communication between the client applications, and as required, communication from various devices associated with the users. Preferably, the backend system is adapted to constantly monitor the communication.

The capability for direct communication between the client devices 13 allows communication to continue, even in the event of a power loss at the backend server 11. In existing systems, when an unexpected event such as a power loss, communication can still occur, but only after a switchover to a backup system, or a switch to otherwise utilise the redundancy in the overall system, is made. This switchover will necessarily cause a downtime in the communication, and can lead to a problem when a mission critical event needs to be immediately reported and addressed. Utilising the presently disclosed technology, the direct communication between client applications running on different client devices avoids this downtime.

The backend system is further adapted to wirelessly receive data measured or taken by one or more peripheral devices 19 in communication with the client device, and/or by one or more instruments 21 embedded with the client device. For instance, if the client device is a smart watch, it may have embedded therein a personal heart rate monitor. A peripheral device 19 may be an instrument, sensor, detection device, appliance, or security apparatus which is linked to the client device.

### Backend Database(s)

To facilitate the integrated service, the backend system 11 includes a database 20 relating to, i.e., provides storage for, various information and management rules needed.

The backend system manages the profiles for the users. There term “user” can mean a client whose wellbeing or activity is being monitored, or one or more other users who are associated with the client (i.e. target user). For example, a target user or client can be a person whose medical status is being monitored. The associated users are those who provide care to the target user or client, such as emergency contacts, family members, medical personnel, etc. A person can be both a target user (i.e. client) and an associated user approved to receive alerts and notifications concerning a different target user. A user who is associated with the client can, e.g., receive notifications or alarms regarding the status or activity of the client, to communicate with the client or other user associated with the client over the messaging application, or to remotely control devices associated with the target client or obtain data from the devices. A client, or a user associated with the client and having the approved access, has the ability to manage—e.g. to update or modify the client's profile information.

A client receiving home care or monitoring will have his or her profile registered with the system. This can be done from the client device 13 via the client application 12, or from the web portal 14 supported by the backend server 11. One or more of the other users will be registered, also via their instances of client applications 12 or via the server portal 14, as an approved party associated with the target user or client. The association or link is stored in the database 20.

The backend server will include at least a user registration database 22 which stores the information relating to the users, and optionally any devices which are registered as being associated with the users. This information will be transmitted from the client application 12, in cases where registration is done on the client application 12 via a user registration interface.

Depending on the user category and the embodiment, the information will include basic personal data (e.g. name, age, home address, contact number, relationship or association with other registered parties who are associated with the user, etc.), professional data if appropriate (e.g. qualification, place of work, care relationship), log-in data (e.g. user name and login key or password, etc.), contact data (e.g. contact information of medical care provider, or a family or friend who is an emergency contact) and associated device data.

The backend database 20 also includes a measurements and detection data database 24, which can be part of, or linked from, the user registration database 22, to store e.g. information obtained from the peripheral devices 19 or embedded instruments 21, such as a heart rate sensor, a blood glucose monitor, a blood pressure sensor, movement sensor, etc.

The backend database 20 may further store monitoring rules associated with the user. The monitoring rules may be stored in its own database 26 which is linked to the aforementioned user registration database 22 and/or measurements database 24, or they may be stored as part of the user information inside the user registration database. 22 The rules may be defined by the client, or may be defined for the client by the client's associated users such as an approved medical practitioner or family member. The rules may define the ranges of expected data (e.g., expected measurements) from the peripheral devices 19 or embedded instruments 21, or the frequency or timing of certain events which the monitoring device(s) are expected to detect.

For instance, the rules may direct that an alarm be sent to a family member or another associated user, if the movement sensor does not detect any movement for more than a predefined period, in a predefined duration of time in a day. The monitoring rules are configurable by the target user and/or at least one or more of the associated users, via the backend portal, or from the client device via a user interface.

Optionally, the database 20 further includes or is associated with an events database 28, where past notable events (e.g. medical emergency events or occurrences where the rules were breached) are stored.

### Backend Controller/Processor

The controller 16 of the backend system, e.g., a processor, includes executable code to support the web-based interface or portal 14, where users can register themselves, or log in to register someone else, manage the service level provided to a client, modify the personal or rules information mentioned above, or look up records of past data or events.

The backend system 11 further includes a communication module 30, to wirelessly receive the sensor or monitor data from the peripheral devices 19 or embedded instruments 21 in real time.

For example, when the processor of the backend system 11 receives, from the mobile application, updates relating to the user's personal information or other account settings, it allocates a memory location in which the data is saved. For instance, the data may relate to one or more monitoring rules for a client. When the backend system receives a measurement data which breaches at least one of the rules, the communication module of the backend system will send an alert to one or more recipient devices of the associated users, or the client device.

Preferably, the controller or processor includes or is operatively connected to an artificial intelligence module 32. The artificial intelligence module 32 has access to the user databases 20 mentioned above, or at least the measurement database 24 where the sensor and monitoring data is stored. The artificial intelligence module 32 determines or infers, from past data available from the measurements and detection data database 24, an expectation or prediction of what the health data or movement pattern should be. The artificial intelligence module 32 receives the real time sensor and/or monitor input received by the communication module 30, and interprets the real time data to determine whether an emergency has likely occurred, and automatically initiates an alert or alarm. The alert or alarm is sent via the communications module 30 (which enables communication via Wifi, 3G, 4G, 5G, or a local network such as the Ethernet), or by a messaging module 34 which employs a different communications protocol than the communication module 30.

Further the artificial intelligence module may determine that the received data indicates a concern which should be addressed, and cause the communication and/or messaging module 30, 34 to transmit or broadcast a message to the client and/or the client's care provider(s). Depending on its complexity, the controller 16 can be localised on one computer, or distributed between two or more computers.

The interpretation by the artificial intelligence module 32 occurs before the real time data is stored into the backend database(s) 20, 24.

### Backend Portal

The backend portal 14 is provided by the backend system 11 as a means of access by users registered with the system. In an embodiment, depending on the profile of the registered user, the backend portal 14 which is presented to the user is different. In one example implementation, the user is asked, at the time of log-in, to identify themselves as a client, a family member, or a service provider such as a medical professional. The web portal presented to a client or their family may be a “family portal” where communication between family members may be private. A client's vital signs data may be posted to the family portal, dependent on preconfigured rules in the client's profile.

The level of access to the backend databases 20, 24 will preferably depend on the user's profile or registration level, and the access control is preferably configurable. A medical practitioner, for instance, may not have access to update the personal information such as birthday or contact data, of a client user under his or her care. A family member of the client, for instance, may have approved access to modify monitoring rules in the rules database 26. In some embodiments, the level of access may decrease from client, family of the client, medical professional, emergency contact, etc.

### Client Application

The client application 12, as mentioned above, is downloadable to a client device 13. It contains computer readable instructions executable by a processor of the client device 13. The instructions may be implemented as program modules, i.e. functions, objects, program interfaces, memory structures, etc.

The client application 12 includes a widget 36 which preferably remains running in the background, even when the client application 12 has not been launched by the client. The widget 36 allows the automatic launching of the application and/or enables particular functions in the application 12 to be triggered automatically.

In one embodiment, the widget 36 is provided to enable the client application 12 to be voice activatable, that is, launched upon the client device 13 detecting predefined voice commands. Depending on the voice command, the widget 36 may start a particular client interface (such as the emergency interface if it detects the spoken command “help”).

In one embodiment, the widget 36 monitors incoming readings of the client's vital signs from a peripheral device 19 or built in instrument 21, and causes the messaging component 34 of the client application 12 to automatically transmit, e.g. broadcast, alarms or alerts to associated users (as shown by the arrows with the dashed lines), when the vital signs readings indicate an emergency situation.

In some embodiments, the client application 12 can have capability to remotely configure the memory or database entry for the corresponding client, in the database. This type of implementation provides a “light” application that imposes a reduced demand on the client device 13, compared with an application which requires more data storage in the client device 13.

For instance, the client application 12 includes executable code which causes a processor of the client device 13, or a processor of the backend system 11, to allocate a memory location to store one or more rules (e.g. health parameter monitoring rules, rules relating to expected movements, etc.). The messaging module of the mobile device or a communication module of the backend system is adapted to broadcast an alert message to at least one of one or more recipients, when it receives a measurement or detection data which breaches at least one of the one or more rules.

The client application 12 includes executable codes that implement several user interfaces as described below. The codes which implement the below interfaces are provided in a single module or reside in different modules.

### Interface(s) for User Registration and Alert Rules

The client application 12 includes an interface for the user to register with the backend server 11, and to provide or update his or her information. The client application 12 also includes an interface for the user to enter or modify rules in relation to circumstances where alarms need to be initiated, and to whom. In this case, the client application 12 will cause a memory location within the client device 13 to be allocated to store the rules. This is because in the most preferred embodiment, the client application 12 is adapted to provide an alarm or alert directly to the approved contact(s), without the alert being routed through the backend system 11, when an exception to the rules occurs.

The client or an associated user can also log into the portal 14 provided on the backend server 11, to update information relating to the client. Examples include personal information relating to the client, or the rules associated with the client. Optionally, the backend server 11 will communicate this change to the client application 12 on the client's device 13, and provide an alert that there has been an update in the rules. The client or an approved user can then authorise the download of the data. In some embodiments, the backend server 11 automatically causes the client application 12 to update the rules, where the client application accesses the data receiving function of the client device 13 to receive the update.

### Messaging and Contact Interfaces

This interface generally allows for creating, sending, and receiving text-based messages to one or more recipient devices of approved users who are associated with the client. It also allows for the initiation or acceptance of an audio-video communication session with an approved user.

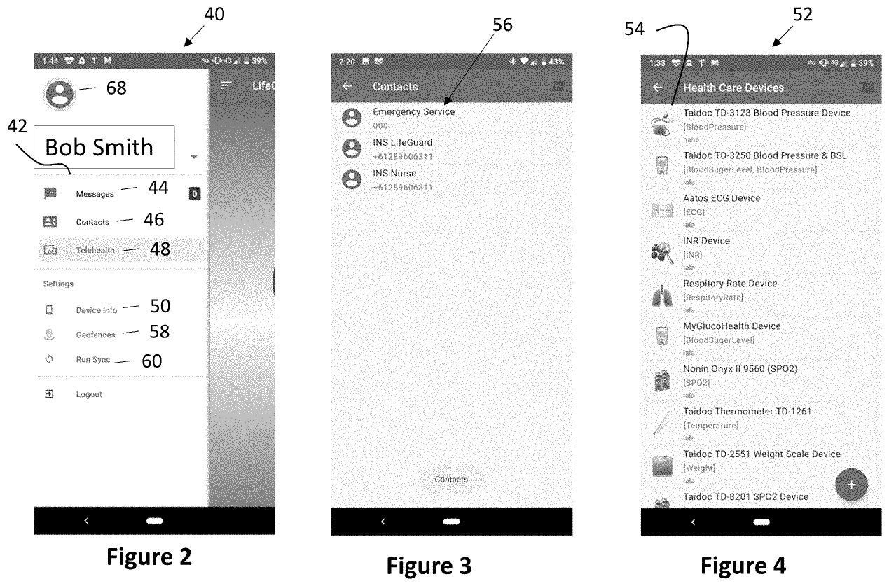

FIG. 2 depicts a menu interface 40 presenting a menu 42 on a display of the client device 13. Here the user can select different functions or select to see different information regarding the account. The menu may be provided as a side bar to an active display or another interface. The menu items are provided as texts, graphics, or both.

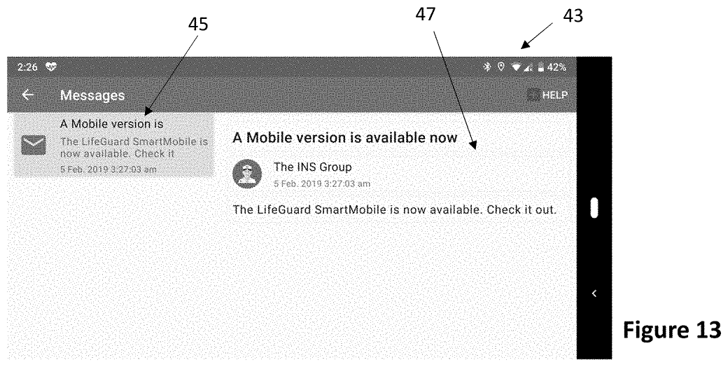

A “messages” menu item 44 enables the user to access the messaging function within the application, to view the messages which have been sent or which have been received. An example of a message display interface 43 is shown in FIG. 13, where a first portion 45 of the display lists the messages, and another portion 47 of the display shows a particular message chosen for display.

A “contacts” menu item 46 enables the user to access a contact list stored in the memory of the client device 13, and also access the messaging function to send a message to a contact. For instance, when the user interacts with the “contacts” menu item, the client application launches a contact list 56, an example of which is shown in FIG. 4.

By interacting with the contact list to select a particular contact, the user accesses the capability to broadcast a message from within the application to the selected contact. The contact list shown includes a public emergency number such as “000”, a nurse, and a healthcare provider's emergency personnel.

A “telehealth” menu item 48 enables the user to contact a telehealth service, to remotely consult a care provider such as a nurse, a general practitioner, or a specialist. The user is able to initiate an audio-video communication session with the care provider, via the audio-video communication supported by the client application.

Although not shown, a “devices” menu item enables the user to see the measurement, monitoring, or sensor devices, or any other tools or appliances which are paired to the client device 13. By interacting with the displayed list, for example via a touch screen, or a physical means on the client device 13 such as a wheel, scroller or one or more buttons, the user is enabled to select a device from the list of devices, and view information regarding the selected device, control an operation of the selected device, or change a setting on the selected device.

### Devices Interface

FIG. 3 depicts a devices interface 52 which is launched when the user interacts with the “devices” menu item. The devices interface 52 includes a display list portion 54 which displays the devices, tools, and/or appliances, which are linked to the client device 13.

In some embodiments, at the target user's location there are located one or more monitoring devices which measure physiological metrics, such as but not limited to, a blood pressure monitor, a blood sugar level monitor, ECG device, international normalised ratio (INR) monitoring device, a respiratory rate monitor, blood oxygen saturation monitoring (SpO₂) device, a thermometer, etc. There can also be one or more other monitoring devices, such as a motion detector in a smart watch worn by the client, or built into the client device 13 itself, an infrared sensor installed at the client's location to detect motion past certain checkpoints, etc. These devices are paired with the client device 13 in that they provide their data to the client device 13. They are alternatively a part of the client device 13, e.g. a heart rate monitor which is provided as part of a smart watch.

The client application 12, which constantly runs on the client device 13, receives the data as it is being measured (see the solid bi-direction arrows in FIG. 1). In an embodiment, the client application 12 determines whether the received data breach any of a predefined set of built in rules (e.g. vital signs rules), and takes action (e.g. launch emergency interface or send an alert to an emergency contact) if there is a breach. For example, if the monitored heart rate increases or drops beyond a threshold, it automatically transmits an alert over the communication protocol to an associated user, such as a nurse, a doctor, or a family member. The transmission of the alert may also go through the backend system.

The client application 12 is adapted to access or be allowed access to the data transmission/receiving capability of the client device 13, and send the measurement and/or detection data from the linked devices to the backend system 11. The data is sent to the backend system via., e.g., the WiFi network, to be stored in the client's entry or entries in the backend database 20. The records of the data are available to be looked up by the target user or an associated user later.

The menu shown in FIG. 2 also includes menu items such as a geofence menu item 58 or client device information item 50, relating to the settings or information regarding the client device or the client's preferences.

A “run sync” menu item 60 can be provided to enable the software or data of the application 12 to be updated from the backend system 11. When the user interacts with this menu item 60, the “run sync” function is activated. The “run sync” function causes the application 12 to communicate with the backend system 11, to check for any updates or messages, or to deliver any messages which is currently on hold in the client application 12 to the backend system 11. Its function is similar to the “send\receive” function on email applications or programs. Whilst the client application 12 and the backend system 11 are constantly communicating, there are some functions that are scheduled (e.g. scheduled checks for software updates). The run sync function forces all those executions to occur manually.

### Information and Settings Interfaces

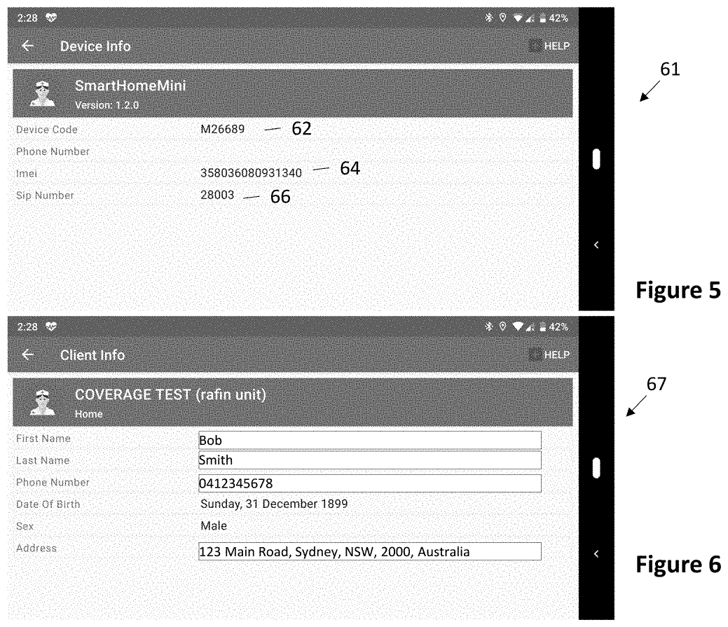

FIG. 5 depicts an example of a device information display 61 which is launched to show information regarding the client's device. It shows, for instance, a unique code 62 assigned to the client device 13, an IMEI (International Mobile Equipment Identity) number 64, a unique identifier of the client device 13 under the messaging protocol 66 (e.g. a Session Initiation Protocol number), etc.

FIG. 6 depicts an example of a client information 67 display which is launched when the user interacts with a “user” item or icon 68 (see FIG. 2). It shows information such as the user name, number, date of birth, and address.

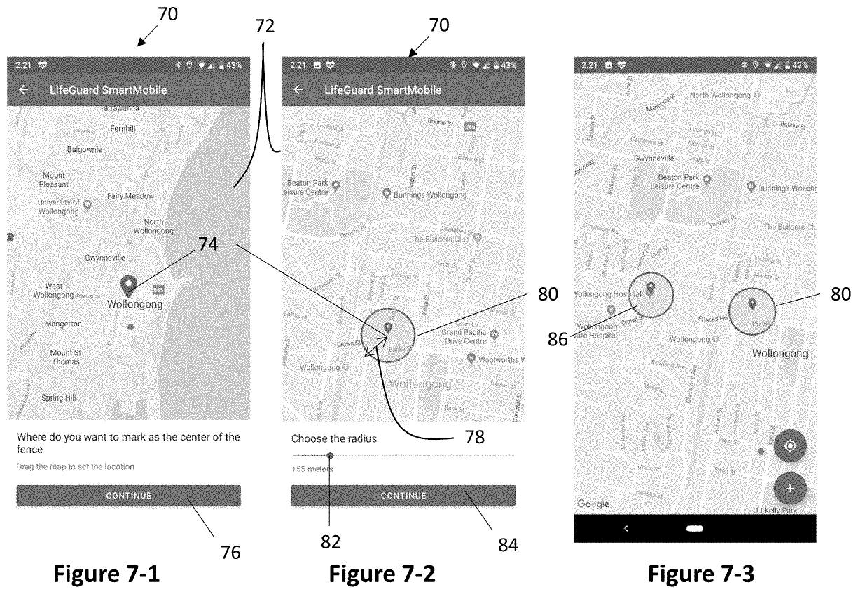

In the menu shown in FIG. 2, there is included a “geofence” menu item 58. The user interacts with the menu by pressing the “geofence” menu item 58, to launch a Geofence settings interface 70, which is shown in FIG. 7-1, FIG. 7-2, and FIG. 7-3. The “geofence” defines an area where the user is expected to be. For instance, in the case of an elderly person with dementia, his or her carer or family member may wish to be notified if the user has wandered too far away from home. Thus, if the user is detected to have left the geofenced area or crossed the “geofence” boundary, an alert is sent to the user's carer. This function requires that the client device 13 includes geo-location or positioning capabilities, such as GPS. The user's carer or family member may also receive an alert when the user is approaching the geofence boundary.

FIG. 7-1 depicts the geofence setting interface 70 when it is first launched. At this initial stage, the interface displays a map 72, and prompts the user to select a centre 74 for the geofenced area by dropping a pin. The centre is, for example, the home address of the user. The interface includes a user input portion 76 where the user can touch the portion or swipe a finger across the screen to confirm the selection. Additionally or alternatively, the interface may prompt the user to activate or press a physical button provided on the device (if available) to confirm the selection.

FIG. 7-2 depicts the geofence setting interface 70 after the user has confirmed the selection of a centre 74 for the geofenced area. The interface now prompts the user to select a radius 78 from the selected centre 74 to define the geofenced area 80. In the depicted embodiment, the interface presents a scrollable portion 82, such as a bar or a button which is scrollable across a line, ruler, or scale, corresponding to a radius range from a minimum distance (e.g. 0 metres or another minimum value) to a maximum distance (e.g. 15 kilometres). As the scrollable portion is moved, the interface displays the distance which corresponds to the position of the scrollable portion. Further, as the scrollable portion is moved, a geofenced area 80 is dynamically changed in the map 72, where the radius 78 of the geofenced area 80 changes as the scrollable portion 82 is moved, and the geofenced area 80 accordingly becomes larger or smaller. The interface 72 further comprises another user input portion 84 to confirm the radius selection and continue to the next step in the setting of the geofenced area.

Multiple geofenced locations can be defined. For instance, as shown in FIG. 7-3, one geofence area 80 is defined around the client's place of residence, and another geofence area 86 is defined around a hospital.

Irregular or arbitrary geofence areas may also be defined. For instance, the geofence area may be defined by street boundaries (i.e., bounded between certain streets).

### Emergency Interface

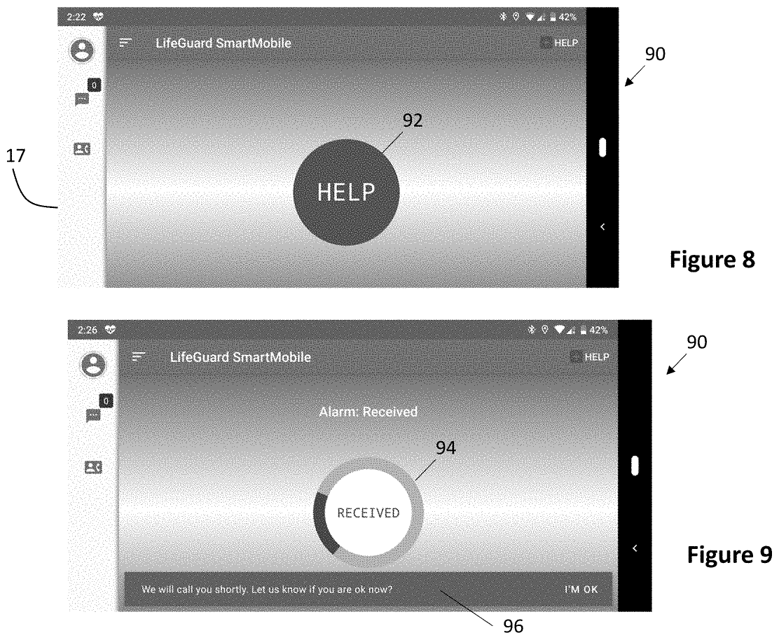

FIG. 8 depicts an emergency interface 90 provided by the client application 12. In this embodiment, the client device 13 includes a touch screen 17. The emergency interface 90 presents a “help” button 92 on the touch screen display, which is preferably prominently sized and located on the touch screen 17. When the user touches the “help” button 92, the application 12 sends an alert or alarm to one or more of the associated users. Once the client application 12 initiates the alert or alarm and the alarm has been received, the emergency interface 90 updates its display to confirm the receipt of the alarm (to show that alarm has been received).

The alarm is generally sent to the integrated service provider or manager, or a service concierge. However the alarm can be sent to another party depending on the emergency level or the event which has activated the alarm.

An example of the updated display 90 is shown in FIG. 9. The updated display includes a text or graphical display 94 to show the client that the alarm has been received. It is preferably shown in a different colour than the previously displayed “help button” 92, preferably one commonly associated with safety or considered soothing, such as green or blue. Optionally, at this stage, the interface 90 includes a confirmation field 96, which as an example is presented on the display as a text, to ask the user to confirm if he or she is okay. In the depicted example, the interface presents a confirmation bar 96 on the display and prompts the user to touch the button if he or she is okay. The confirmation field 96 may be configured to be accompanied by, or may be replaced by, an audio request for the user to confirm if he or she is okay. If an audio request for confirmation is provided, the interface will also be adapted to receive audio input from the user to provide confirmation.

Upon receipt of the alarm, the emergency personnel can take one or more of several actions. They can for instance start the in-app audio/video communication to communicate with the user, remotely control a sensor to check the user's vital signs and review the data, ask the user to take a physiological measurement if the user is able and review the measurement(s), dispatch emergency help or an ambulance to the user, dispatch security personnel to the user's location etc. These functions are enabled over the communication network transmitting the data traffic between the client device 13 and the backend server 11, where the remote user transmits a command which is received by the backend system 11, and the backend system 11 transmits the command data to the client device 13. Or, the remote control command is directly transmitted between client devices 13 via the messaging function of the client application 12. The alarm and update of events are preferably directly broadcast to other emergency contacts, such as family members, via the in-app messaging system.

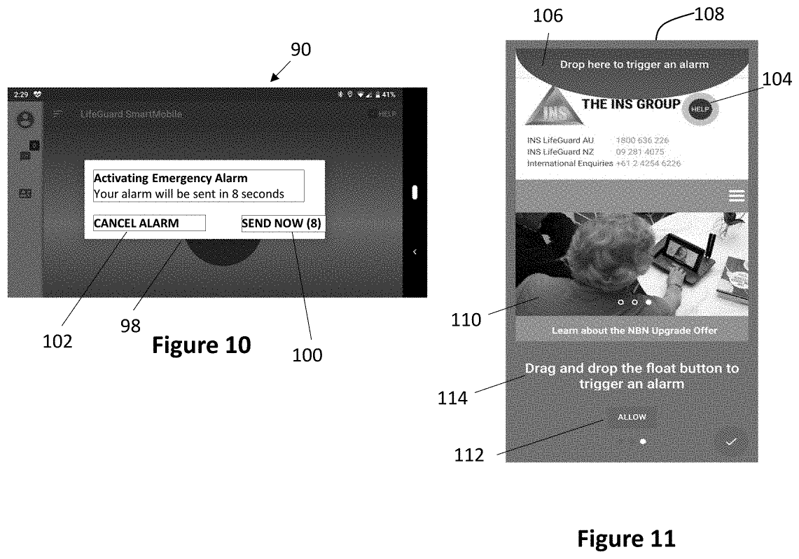

As an option, in some instances, after the emergency interface 90 is launched, but before an alarm is initiated and transmitted from the client device, the interface 90 prompts the user to confirm if an alarm should be sent. An example is shown in FIG. 10. Here, the interface 90 is adapted to cause the screen 17 to display a pop-up confirmation box 98 which prompts the user to confirm whether an alarm should be sent, or cancel the alarm. The confirmation box 98 includes or indicates two areas 100, 102 where the user can interact with the interface 90. The user touches one of the interactive areas (or activates a first button) 100 to confirm that an alarm should be sent, and the client application 12 causes an alarm to be transmitted. The user touches the other interactive area (or activates a second button) 102 to cancel the alarm. Optionally, the launch of this confirmation box 98 starts a time countdown (e.g. from 10 seconds or any other predetermined time period), and at the finish of the countdown the alarm will be sent even if the interface 90 does not detect a user interaction with the confirmation box 98.

The application 12 triggers the emergency interface 90 to be shown on a display or screen on the client device, when it receives a signal that there is an emergency. Depending on the particular embodiment, the emergency interface can be triggered in one or more different manners.

In one example, the emergency interface 90 is voice activated. The widget 36 (see FIG. 1) running in the background monitors for particular voice commands, and activates the emergency interface when it receives the commands. The client application 12 is adapted to receive sound or voice data from a microphone provided on the client device 13, and is adapted to monitor for predefined spoken keywords or phrases, such as “help”, “emergency” etc. When the client application recognises one of the keywords or key phrases, it launches the emergency interface. The emergency interface 90 may further have access to the audio input and output of the user's device, so as to provide the prompts to the user as audio prompts, and to receive the user's audio input as confirmation to send the alarm or cancel the alarm.

In some embodiments, where the client device employs an Android system or another compatible system, the client application will utilise built in voice assistant features such as those provided Google®.

Depending on the hardware availability, the client application in some embodiments is adapted to receive a signal from a switch or button, such as a capacitive button or a push button, located on the client device. When the client activates the button, the application 12 launches the emergency interface 90.

A further manner of triggering an alarm or the launch of the emergency interface is depicted in FIG. 11. In this method, the client application 12 provides a float button 104, and a destination area 106 for the float button 104. To launch the emergency interface 90 or to directly trigger an alarm (depending on the specific implementation or user setting), the user drags the float button 104 to the destination area 106. The float button 104 and the destination area 106 are provided over a background, which can be an interface which is being launched or which is currently shown on the display.

In FIG. 11, this background is a “home page” or initial display 108 presented to the user at the launch or the initialisation of the client application 12. In the depicted initial page 108, there is a scrollable display portion 110, where the user can scroll through to see general information regarding, e.g., the client application and its function, the telehealth provider, medical services, offers by other service providers, etc. FIG. 11 further shows that, in the depicted embodiment, at the set-up stage, the user is asked to allow the “drag and drop” function to trigger an alarm, by pressing an “allow” button 112 under a description 114 of the “drag and drop” function.

The emergency interface 90 may further be launched automatically or the alarm is triggered automatically, when one or more instruments 19, 21 (e.g. sensors) embedded in the client device 13 or peripheral devices paired with the client device 13 detect a critical event, such as a possible heart attack or a dangerously low blood glucose level. Detection of the critical event will be dependent on detection of whether the data from the one or more instruments breach one or more critical rules or conditions. In another example, the emergency interface 90 is automatically launched, when a movement sensor (e.g. an accelerometer) detects a fall, but does not detect that the user has gotten back up within a predetermined time frame. The automatic launching is either triggered by a predefined set of trigger rules as part of (i.e. built into) the client application, or triggered by the client application which is in communication with the backend to “listen” for a backend system determination that an emergency has occurred.

The client application is thus enabled to provide a suite of active care or passive monitoring functions with both single direction and bi-directional communication capabilities.

### Pattern Recognition/Adverse Event Recognition

As mentioned above in relation to the system's architecture (an example is shown in FIG. 1), a controller 16, preferably one employing artificial intelligence 32, is provided at the backend 11. The controller 16 monitors the data from the peripheral devices 19 or embedded instruments 21, which have been transmitted from those devices or instruments 19, 21 to the client device 13, and then transmitted by the client device 13. The controller uses predictions made by the artificial intelligence 32 to detect emergencies or potential adverse events. In the event that a potential problem is determined, the controller causes the adverse event to be broadcast to the associated user, as an alarm or as a message, depending on the severity of the issue. Preferably, to avoid a delay in the determination of an emergency event, incoming data is first processed by the artificial intelligence before being saved into data storage.

For example, the controller monitors the data from the associated devices over time, and interprets whether there is a pattern or change which is of concern. If so, it sends an alert or a message to one or more of the users associated the client or target user. For example, the controller may send a message to an associated user (e.g. a doctor or a family member) if it detects, from the movement data received over a period of time (e.g. a month, 6 months, or a year, etc., as can be determined by the skilled person depending on the relevant circumstances), that the amount of movement detected has been gradually decreasing over that time.

### Telehealth Consultation

The application enables the client to consult a health care professional remotely. The bidirectional communication enables the two-way audio-video consultation and conversation between the client and the health care professional. The health care professional will be an associated user having access to their own client device, or having access to the system via a web portal linking to the backend server.

The health care professional is able to direct the client to take a health measurement (e.g. heart rate, glucose level, temperature) from a registered sensor or monitor device. The healthcare professional can also direct the client to set up a particular sensor or monitor device, i.e. by placing electrodes on the required locations on the client's body, and then initiate the operation of the sensor or monitor device. The data from the sensor or monitoring device is transmitted wirelessly to the health care practitioner in real time. As the practitioner is an approved user of the system, depending on their access level, they will further be able to direct alarms or alerts to be initiated, or review past data relating to the client. Thus the health consultation capability enabled by the management system extends beyond simply enabling an audio-video verbal discussion with the practitioner.

### Remote Sensor or Monitor Operation Control

The sensors, monitoring or measurement devices 19, 21 are typically operated by the client or someone else who is on location to operate them. The client application provides the software, and if required, user interface, to control the connection with these sensors, monitoring or measurement devices 19, 21. However, the client application also allows an associated user to remotely initiate and/or control a monitoring or measuring operation, to take vital signs of the client. This remote control is possible where the operation of the monitoring device is automatic or does not require additional client action. For instance, the device may be a wearable heart rate or body temperature monitor which is worn by the client.

As mentioned previously, the client device is caused by the client application to save the data, and to transmit it to the backend system 11 for analysis. The backend system 11 will take further actions if required, on the basis of the received data.

Thus the bidirectional management system allows a scenario, where an associated user receives an alert from the client application, and remotely operates a monitor or sensor, to check e.g. a vital sign and to assess the situation.

The associated user, typically a nurse or another health care professional, may also remotely control the operation of the peripheral device 19 or embedded instruments 21, where the client has set up the sensor or monitor device ready for operation.

This is useful in cases where the user has diminished stability in his or her limb movements, or reduced digital flexibility (e.g. in the fingers), and needs assistance to operate the peripheral devices linked to the client device.

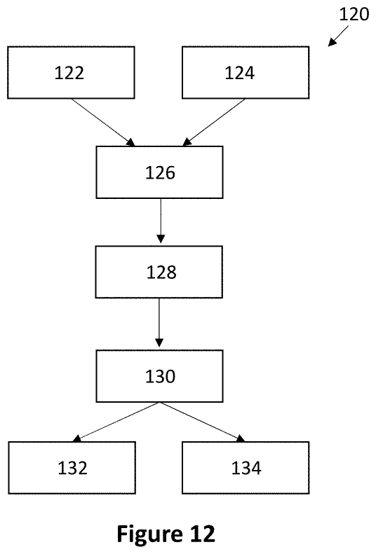

FIG. 12 depicts an example scenario 120. A sensor or monitor monitors the client's physical activity 122, and a passive motion detector (e.g. an infrared detector) monitors certain movements (e.g. client moving through a bathroom door) 124. The data from these devices are paired to the client device, and then transmitted by the client device to the backend 126. From these data, the backend server determines whether there is a problem event, e.g. by comparing the received data with the alert rules 128. A problem event occurs, e.g. when the backend server determines that there is an abnormal movement (e.g. a motion sensor detects a fall without subsequent detection that the client has gotten back up), or determines that there is a missing movement which should have occurred in a certain time frame (i.e. an infrared sensor 106 detecting a first motion through a bathroom door but does not detect another motion through the door for more than a certain amount of time) 124.

If a problem event is detected, the backend server 11 communicates with the client application to initiate an alert 130. The alert is broadcast to the devices of one or more associated users (e.g. a health care staff or a family member) 132. The associated user then uses his or her client device to remotely control, e.g., a heart rate sensor worn by the monitored client, to obtain a heart rate measurement 134. Alternatively or additionally, the associated user can, from the client application installed on his or her device, initiate an audio-video communication session 136 with the device of the monitored client, so as to contact the client.

### Bi-Directional Messaging

As mentioned above, from within the application, the client is able to launch the messaging interface and/or a contact list, to select a contact and call the contact or send a message to the contact.

The messaging protocol also allows an alarm to be broadcast to one or more of the users associated with the client, in case of an emergency. This can be triggered by an emergency interface being launched, or automatically by the application when it receives sensor data that indicates an emergency or a catastrophic event.

The messages are transmitted to the contact using the application's messaging protocol, if the contact is an associated user who also has the application running on their device. Similarly, the application is adapted to receive a text message from or an audio and/or video call from another user (e.g. such as a care taker needing to make an appointment with the client to check the status of the client). The messaging protocol enables audio-video messaging between client devices. The messaging protocol enables an approved user (e.g. a general practitioner, an approved service provider etc.), to log onto the web portal and contact the client via audio/video messaging.

The application is also adapted to receive an alert or alarm. For instance, an associated user (e.g. a family member) can use his or her instance of the mobile application, or log into a web portal, to send an alarm or an alert to the client to remind them of the time to take their medication. The client or an approved associated user can also log into the portal (or via the mobile application) to provide a rule that an alert to take medication be sent at certain times, or on a particular schedule.

In a further embodiment, the backend system is able to broadcast a message to all users in a particular geographical location. For instance, the backend system will broadcast an alert or message to all users in a particular geographical location or area where the air quality is low (e.g. affected by bush fire and windy conditions) to stay indoors.

The transmission is preferably encrypted to protect the client's privacy.

In further embodiments, the application is further adapted to communicate with other communication platforms (e.g. voice over IP telephony platform, mobile data platform, etc.) to communicate with personnel over those platforms, and/or to initiate a call to the selected contact number if mobile data or a wireless network is not available.

### Passive Monitoring

As mentioned above, the measurement or detection devices which are associated with the client can be provided in the surrounds populated or inhabited by the client, such as their own home. This may include motion sensors placed in locations such as the front door, the bathroom door, near the refrigerator, etc. These detection devices are registered to the client, and their data is paired to the client's device to be transmitted to the backend server.

In an embodiment, the client application and/or the backend server will broadcast a message to the client's carers or family that there is a need to check on the client, from the detection data. For instance, such a need may be determined when a motion sensor does not detect an expected motion within a reasonable time frame (e.g. only a single motion through a bathroom door in two hours). This need may also be determined when a motion sensor does not detect an expected motion within a certain time period in the day (e.g. no detection of motion past the bedroom door during the hours where the client is expected to wake up and start the day).

### Home Automation/Security Management

The devices which are associated with the application need not be only vital signs sensors or monitoring devices. They can further include home appliances or control switches, such as but not limited to lights, air conditioners, heaters, fans, televisions, power switches, and security appliances or equipment such as intercoms, door and/or window locks, etc., which are paired with the client device, e.g. via Bluetooth®, or are linked to the same wireless network with the client device. The client is enabled to remotely control the paired or wirelessly linked devices via the “devices interface” of the client application or by logging into the web portal. An associated user, such as a family member of the client, can log into the web portal to control the paired devices in the client's home.

The client application, in conjunction with its data and control integration with the various health or security monitoring/sensor devices, enable an integrated management system for health and security management, and home automation.

For example, using the client application (or via the web portal), a client or a family member user can remotely operate security apparatuses (e.g. to unlock the door locks), when they receive an alert that there has been an emergency alarm and emergency respondents are on the way, to ensure the respondents have unobstructed access to the patient.

In the above, the various client devices—of the client or other users associated with the client, and also any other sensor or detection devices or instruments which communicate with the client devices, form an eco-system. The devices in the eco-system together provide a cohesive management system for the client, in relation to their health, safety, security, comfort or convenience (e.g. home automation).

### Eco-System Including At-Home or Out-Of-Home Devices

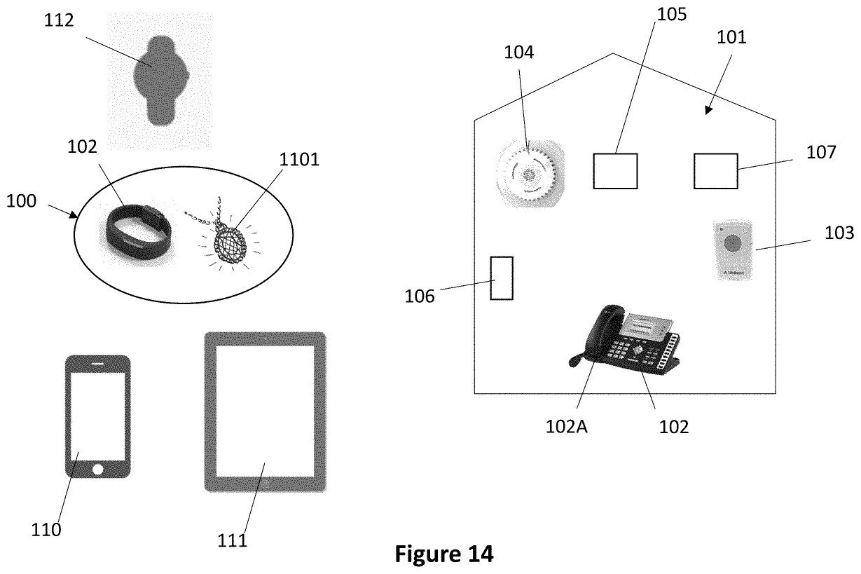

FIG. 14 illustrates a home care, security and home automation system in accordance with an embodiment of the present invention, generally designated by reference numeral 101. The system builds on the system of applicant's earlier patent application (referenced above) and includes some further features, as will be described in detail in the following.

The system includes a central communications console or “hub” 102, which is arranged to communicate with peripheral devices in the home. The hub 102 also has a remote communications facility to communicate with a remote location. The remote location may, for example, comprise a base station (not shown) which can communicate with carers or service providers, such as nurses, doctors, family members, so that they can communicate with the client via the hub 102. Alarms can further be sent to the base station. In accordance with the referenced patent application, the base station can implement video communications and voice messaging. There is also a telephone handset 102A associated with the hub 102.

The hub 102 includes a short range (e.g. Bluetooth4™ or wireless) communications arrangement or process for communicating with devices in the home, i.e., in-home or on-premise devices. In this system, the devices may include any device useful for monitoring and maintaining health, security, home automation, and any other aspect of care, for the client.

The devices may include:
One or more call point devices 103. Such devices may be used to send an alarm back to a remote location via the hub 102. Call point devices 103 may be placed at various about the house or premise where the hub 102 is located.
One or more health monitoring devices 107. These may include blood glucose meters, blood pressure monitors, ear/forehead thermometers, weight scales, hand held ECGs, pulse oximeters, and more. They may use local communications to communicate data to the hub 102, via which the data is communicated back to the base station, for storage and monitoring.
One or more environmental alarms 104 such as smoke alarms 104 and other alarms. As well as providing an alert (sound) function, these may include local communication modules to communicate to the remote location via the hub 102, to provide an alarm to the remote location.
One or more motion detectors 105, adapted to monitor the motion of a client, so that remote monitoring of motion can be carried out to ensure that the client is active, for example. This is also useful to monitor expected movements of the client. For instance, signals from the motion detectors can be used to monitor whether the client leaves his or her bedroom in the morning.
One or more home automation or security sensors 6. These can be used to control home devices or appliances, such as lights, temperature (thermostats), etc. Control can be implemented from the hub 2 or even remotely from the base station via the hub 102.
Other devices that may implement one or more forms of health, security or home automation.

The above described system is provided mainly for use in the home or a premise where the client is staying. Many clients are mobile, however, and it would be useful if monitoring devices could be provided that could leave the home and still be functional.

Some Apps for monitoring clients are known for upload to mobile devices, such as smart phones 110, tablets 111 and smart watches 112. Simple Apps are known for these devices such as alarm Apps, for example. These devices 110, 111, 112, however, are genuinely arranged to have a remote communications facility for communicating directly with a remote station. These devices 110, 111, 112 will use this remote communications facility, more often than not, for communicating the alarm. This results in relatively a low battery life for communications.

In accordance with an embodiment of the present invention, some or all of the devices of the system, as well as the usual “outside home” or mobile devices 110, 111, 112, are fitted with a local communications arrangement, arranged to communicate with other devices in the system. This local communications arrangement may provide short-range communication (e.g., Bluetooth), or extended local communications, such as Bluetooth 5 standard (BT5) or above which provides short range and extended range communication. For example, a mobile device 110, 111, 112 outside the home may have extended range to communicate with the hub 2 (also having BT5, for example), and then the hub 2 can communicate the data back to the remote location (e.g. base station). This has the advantage of not requiring heavy use of batteries to support the long-range communications technology. In embodiments, mobile devices 110, 111, 112 (or other devices that may be used outside the home, such as health monitors 107, etc.) may have implemented thereon a process for locating a compatible device via the local communications arrangement. If a compatible device can be located, it will pass the communications to that device, rather than using the long range communications technology.

Another aspect of the system is that devices 110, 111, 112, with long range communications, may be contacted by a local communications device of the system (e.g. health monitor 107) to communicate data back to a base station, instead of the hub 102. Health, security, or environmental, or other data reading, could therefore be taken at or from the home. For example this will include the health data as read by a health monitor or sensor 107 which has local communications capabilities only. The data can then be communicated to the mobile or “out of home” devices via local (short range or extended short range) communication. Then, from the mobile or “out of home” devices, the data can be communicated back to the base station via remote communication, or where appropriate, extended long range communication. Other devices could be operated in this way, such as the home automation devices or appliances within the home, by devices 110, 111, 112 outside the home.

An embodiment of the invention also includes an alarm device 100, which may be carried outside of the house and worn on the client's person. It may be provided in the form of a pendant 101 or a wrist bracelet 102. The alarm device 100 includes a local communications facility, but also a communications process arranged to locate a compatible device (such as a mobile device 110, 111, 112 or the hub 102) with a compatible local communications facility.

Data, alarms, or messages from the alarm device 100 can be sent from the alarm device 100 to the compatible local communications device 102, 110, 111, 112. The compatible local communications device 102, 110, 111, 112 may have a remote communications facility to send the message/alarm back to a base station. Messages may also be sent from the base station to the alarm device 1100, via the compatible device 102, 110, 111, 112. Utilising a local communications facility only, enables life of a battery of the device 100 to be extended. This is one major advantage, as the critical alarm device can be carried, and functioning for a number of years rather than requiring charging. Another advantage, is that the alarm device 100 can detect compatible devices anywhere within its communication range, and implement alarm communications with a base station via the compatible device. The compatible device could be the user's own smartphone 110 or iPad 111 or watch 112, or could be another user's device in the vicinity. All the devices will have a compatible Application installed thereon.

In some embodiments, two or more devices may need to be linked or associated with each other, for the communication between the devices to be permitted. For instance, the devices may be registered to the same user account, or they may be registered to different user accounts that are associated with each other. Data linking may require the receiving device to recognise an identifier associated with the transmitting device, or for both devices to recognise identifiers associated with each other. The determination of these potential relationships or associations may be made on the basis of firmware identification, or by user account identifiers being sent along with the data and being checked by the controllers or processors of the respective devices.

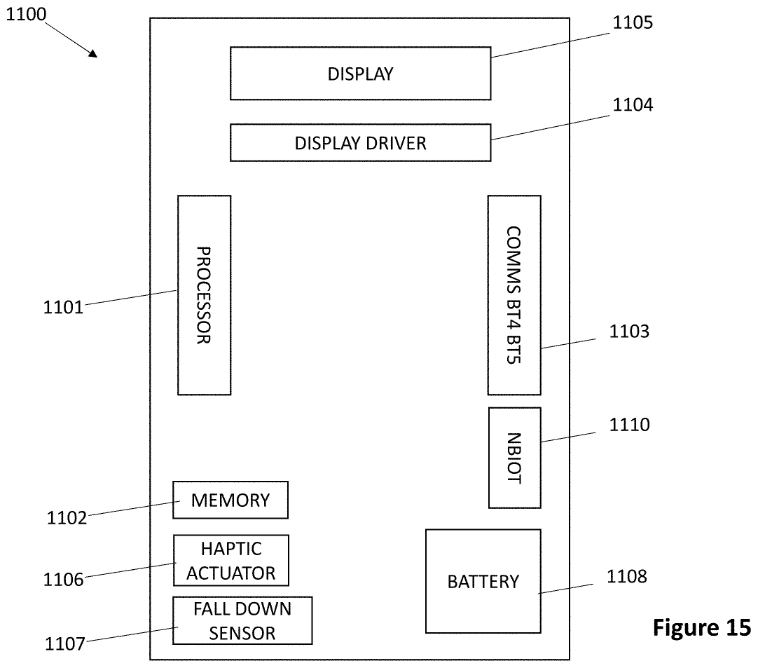

A block diagram of the critical alarm device 1100 is shown in FIG. 15. The device comprises a processor 1101 and memory 1102 for executing computer processes. The processor 1101 is adapted to implement at least an alarm function process, which is arranged to provide an alarm indication. The processor 1101, memory 1102, and other components such as circuitry or circuitries, will be mounted within a housing.

The alarm device 1100 comprises communications circuitry 1103 arranged for local communications. In this embodiment, the alarm device 1100 is enabled with two types of local communications, being short range and extended short range communications. An embodiment of the alarm devices includes capabilities to communicate data (such as the alarm indication) using both BT4 and BT5 protocols. As will be discussed, the processor 1101 may select which communications protocol to use, depending upon the compatible devices that are detected.

In addition, the alarm device 1100 comprises a display driver 1104 and display (e.g., organic light-emitting diode, or “OLED”) 1105. Feedback about the alarm indication or confirmation of the transmission and receipt of the alarm indication, may be provided by the display 1105, and messages may be provided on the display (e.g. “your alarm has been received”). Therefore, the communication capabilities include both receiving and transmission capabilities.

In some embodiments, the feedback about the alarm is only be provided if the alarm has been confirmed to be received by the device of a carer or service provider, or by the base station. In these embodiments, the feedback may incorporate data, such as an identifier or a name of the party or device which has received the alarm.

The alarm device 1100 also includes a haptic actuator 1106. This may be a vibrator, for example, for causing a vibration on alarm or feedback or for another reason.

The device 1100 may include a speaker or another audio component, for sounding or emitting an audio message or alarm.

A fall down sensor 1107 may also be provided. This may be configured to implement an alarm on detecting that the client has fallen.

A battery 1108 for powering the device 1100 is also provided. The battery may have a very long lifetime, facilitated by the fact that the communications from the device do not require a great deal of power. Lifetime may range from two to six years or even longer. This means that the client does not have to be concerned about constantly recharging the battery of their device. The device remains functional for a long time, providing its critical function of alarm monitoring.

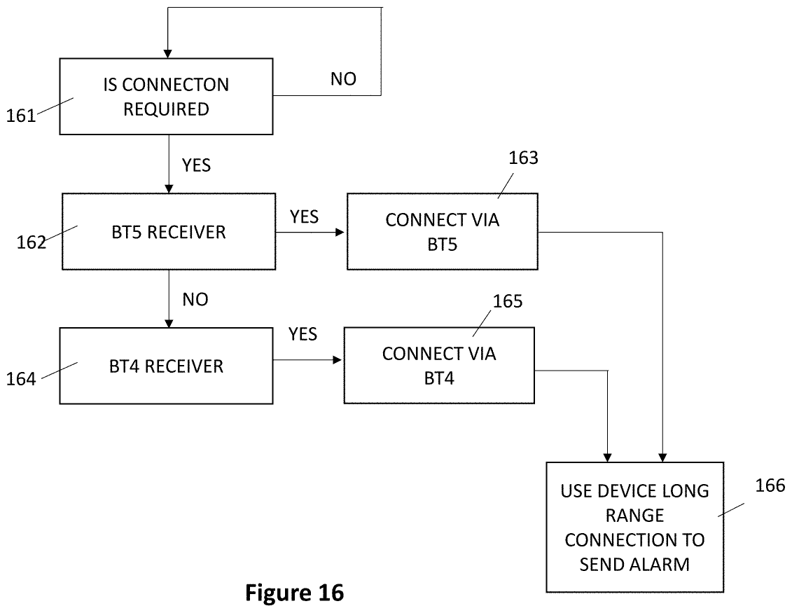

FIG. 16 illustrates a communications protocol process of an embodiment of the device 1100.

At step 161, the processor 1101 decides whether a connection, i.e., a communication link with another apparatus or device, is a required. The connection may be required because an alarm has been implemented, i.e., an alarm condition has been met. For example, when it is determined the signal provided by the fall down sensor 1107, that the client has taken a fall (the alarm condition), this determination triggers the processor 1101 to determine that a connection is required. Otherwise, no connection may be required. Therefore, a determination that an alarm condition has been met, may also provide a signal to the processor 1101 to trigger the establishment of the connection.

If “YES” (i.e., the processor 1101 determines that a connection is required), a location process (step 162 is implemented via the extended range communication protocol (e.g., the BT5) to determine whether a compatible device is in range and has protocol compatibility. If “YES” (i.e., a device with the compatible communication protocol is detected as being in range), then, at step 163, the communications device 103 connects and transmits via the protocol (e.g., BT5), with the compatible device. If “NO” (i.e., no extended range protocol compatible device is in range), the next step 164 is to undertake a process to determine whether a standard short range (e.g., BT4) receiver is in range (step 164). If “YES”, connection is made via the short range protocol (e.g. BT4) in step 165, with the detected receiver, and hence the compatible device having the detected receiver.

It will be appreciated that the connection steps can be altered. That is, the processor 1101 may attempt to detect and connect with compatible devices over the short range (e.g., BT4) protocol, and if then unsuccessful, attempt to detect and connect with compatible devices over the extended range (e.g., BT5) protocol.

When connected to a compatible device, the alarm device 1100 may send data to that compatible device. The compatible device in turn which may then be used for long range communication to a base station to send an alarm (step 166). Feed back may be provided through the alarm device 1100, which can be displayed on a display 1105.

The aforementioned connection, from the alarm device 1100, may be made by way of any compatible device, including other persons' devices (e.g. other person's smartphones having the App installed). This connection may be with the hub device 102, where the person is walking out of the house (in the garden for example).

The devices described above (i.e. the hub 102, call point device(s) 103, various alarms, detectors, and monitors, 104, 105, 106, 107, 110, 111, 112, and alarm devices 1100, 1101, 1102) and other compatible devices may form an “eco-system” for the management of health and security of a client, both in and out of the home.

In one embodiment, the alarm device 1100 is also provided with a narrow band (NB) internet of things communications circuitry 1110. This may connect to a remote location via a relay of the internet of things and NB network. Variations and modifications may be made to the parts previously described without departing from the spirit or ambit of the disclosure.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A mobile application including:
a client application comprising executable codes downloadable to a client's device, the client application comprising a messaging module adapted to transmit messages to one or more recipient devices associated with approved users who are associated with the client, and to receive messages transmitted from the one or more recipient devices; and
a data transmission and receiving module adapted to receive a data from one or more peripheral devices in communication with the client device or one or more instruments embedded with the client device;
wherein the data transmission and receiving module is adapted to transmit the data to a backend system, wherein the messaging module is adapted to function independently of the data transmission and receiving module, and wherein
the backend system is adapted to support a portal accessible by one or more approved users to review the data.

2. The mobile application of claim 1, wherein the messaging module is adapted to broadcast message to, and receive messages broadcast from, the one or more recipient devices.

3. The mobile application of claim 1, wherein: the recipient devices being devices on which there are installed other instances of the client application, or devices which host an access interface to the backend system; or wherein the one or more recipients include an active login session on the portal supported by the backend system.

4. (canceled)

5. The mobile application of claim 1, comprising a widget which is adapted to be responsive to a voice command, to launch the mobile application.

6. The mobile application of claim 1, wherein the backend system comprises a processor which includes or is operatively connected to an artificial intelligence module which is adapted to infer occurrences of emergencies or potential adverse events based on the data transmitted to the backend system.

7. (canceled)

8. The mobile application of claim 1, comprising executable code which causes a processor of the client device or of the backend system to allocate a memory location to store one or more rules, wherein the messaging module of the mobile device or a communication module of the backend system is adapted to broadcast an alert message to at least one of said one or more recipients, when it receives a measurement or detection data which breaches at least one of the one or more rules.

9. (canceled)

10. (canceled)

11. The mobile application of claim 8, wherein the client device has geo-location capability, and the executable code causes the processor to allocate a memory location to store a geofence setting information including a centre and a radius defining a geographical location.

12. The mobile application of claim 1, comprising a plurality of executable program modules to provide a plurality of user interfaces, including one or more of the following:
an emergency interface which is automatically activatable, activatable by the client, or activatable by a command via the backend system, provided by at least one of the approved users;
a geofence settings interface, which enables a definition of a geographical activity area associated with the client;
a measurement or detection devices interface adapted to allow user control of the one or more peripheral devices or embedded instruments;
a user registration interface which enables user information to be entered or updated from the client device;
an information or settings interface for displaying device and/or user setting information, and for updating device information and/or user setting information;
a messaging and contact interface for creating, sending, and receiving text-based messages to one or more recipient devices, or for initiating or accepting an audio-video communication session.

13. The mobile application of claim 1, adapted to receive a control command from the backend system, initiated by one of the one or more approved users, for remote control of at least one of the one or more peripheral devices or embedded instruments.

14. (canceled)

15. A communication system comprising a backend system, in wireless communication with a plurality of instances of client device each having installed thereon a mobile client application as claimed in claim 1.

16. The communication system of claim 15, said backend system including at least one database which are accessible and updatable by one or more of the approved users via their respective recipient device or via a web-based portal supported by the backend system.

17. (canceled)

18. (canceled)

19. (canceled)

20. (canceled)

21. A health and security management system, comprising:
a backend server having stored thereon registration details of two or more users, at least one user being a client whose health and security is being managed;
the backend server being adapted to communicate over a long range communication network with two or more apparatuses, at least one apparatus being a client apparatus registered to the client, the other apparatuses being apparatuses registered to users associated with the client;
the apparatuses each having a processor and at least one non-transitory computer readable medium storing machine-readable instructions for execution by the processor to:
monitor, over the long range network, communication from the backend server which is to be transmitted to the two or more apparatuses;
obtain data from one or more sensors, detectors, or monitors, and transmits the data over the long range communication network to the backend server;
initiate a user interface process when an alert condition is met, the alert condition being met if the obtained data is determined to breach one or more conditions or is determined to satisfy one or more detection rules;
the backend system being adapted to monitor direct communication between said apparatuses; and receive said data from the client apparatus.

22. The system of claim 21, wherein the determination of whether the alert condition is met is made by a processor of the backend server.

23. The system of claim 21, wherein the processor of the client apparatus is configured to determine if the obtained data satisfy an emergency condition, and upon determination that the emergency condition is satisfied, communicate with at least one other apparatus using a direct communication process module.

24. The system of claim 22, wherein the client apparatus is configured to directly transmit an alert to the other apparatuses using respective direct communication process modules implemented on the client apparatus and the other apparatuses.

25. The system of claim 22, wherein the user interface process comprises a process to request an input from the client.

26. A mobile client care device, comprising a housing mounting circuitry including a processor arranged to execute computer processes, an alarm function process arranged to implement an alarm indication, the communications arrangement comprising a local communications process arranged to, after said alarm function process implements the alarm indication, locate any available devices having a compatible local communications process, and communicate the alarm to the available device, whereby the available device may communicate with a remote location.

27. A device in accordance with claim 26, wherein the local communications process is arranged to locate compatible devices that have a corresponding local communications process and also have a remote communications process.

28. A device in accordance with claim 26, wherein the local communications process is configured to communicate over a plurality of local communications protocols.

29. (canceled)

30. (canceled)

31. (canceled)

32. (canceled)

33. A system for client health care and security, the system comprising a plurality of devices, each of the plurality of devices comprising a communications arrangement, comprising a local communications process arranged to locate available devices having a compatible local communications process, for communication, and at least one of the devices having a remote communications process for communicating remotely.
